(19) United States
(12) Patent Application Publication
(10) Pub. No.: US 2024/0041315 A1
(43) Pub. Date: Feb. 8, 2024

(54) Deep leaning artificial intelligence method in predicting personalized healthy original undamaged retinal nerve fiber layer thickness contour/profile using anatomical parameters and optical coherence tomography

(71) Applicant: Ahmad Najafi, Detroit, MI (US)

(72) Inventor: Ahmad Najafi, Detroit, MI (US)

(21) Appl. No.: 17/817,990

(22) Filed: Aug. 7, 2022

(51) Int. Cl. A61B 3/10; G06T 7/00

(52) U.S. Cl. CPC A61B 3/102; G06T 7/0012; G06T 2207/20084; G06T 2207/10101

(57) ABSTRACT

In this invention, we used for the first time GAN method of deep learning in AI to predict the personalized normal undamaged original RNFL thickness contour/profile, using anatomical parameters of peripapillary blood vessel number, size and location from the OCT B-scan images. This is the first time that a personalized RNFL thickness contour/profile will be available and can potentially replace the current so called normative database of the RNFL thickness contour/profile.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### SEQUENCE LISTING

Not Applicable

### BACKGROUND OF THE INVENTION

Glaucoma is a progressive neurodegenerative disease of the eye. It results from loss/compromise of ganglion cells in the innermost cellular layer of the retina. Often times, patients are labelled as “glaucoma suspect” because the ophthalmologist cannot interpret the multimodal imaging tests of the patient with opposing results, as a patient's test result lies “within normal range” of the normative data base while it lies “outside normal range” for other tests.

Optical coherence tomography (OCT) measure of retinal nerve fiber layer (RNFL) thickness is an objective way of diagnosis/surveillance of glaucoma. It is also based on the comparison between a subject's RNFL thickness contour/profile and the “normative data base” RNFL thickness contour/profile.

Normative data base has been the gold standard of comparison for data/test interpretation in medicine. It is formed from the data of “presumably normal healthy subjects”. Although normative data base can help in some clinical settings, it can't be accurate enough to diagnose an abnormality when the range of normality can overlap with that of abnormality, one example being patients with “glaucoma suspect” diagnosis. Therefore, there is a need for a“personalized normal” measure of RNFL thickness to overcome the shortcomings of “normative data base”, as the gold standard of comparison.

In our proposed method, we showed that we can accurately predict the original healthy undamaged “personalized” RNFL contour/profile of each person based on his/her anatomical parameters (APs), using deep learning method of artificial intelligence and OCT imaging technology. In other words, instead of comparing a patient with a “normative data base”, which is formed from a very limited presumably healthy subjects, we can predict with high accuracy the “personalized original healthy undamaged” RNFL contour/profile of a person and use it to compare a patient's measure of his RNFL thickness contour/profile with his own “normative data base”. That is, the person will be compared with his/her own normal values, instead of being compared with “normative data base” of other “presumably normal subjects”.

### BRIEF SUMMARY OF THE INVENTION

This is a proof of concept project. We showed that deep learning artificial intelligence (generative adversarial neural network=GAN) can accurately predict a RNFL thickness contour by using anatomical parameters (APs) of number, size and location of peripapillary blood vessels, derived from an OCT B-scan. All data used in this project are computer generated. Currently, normative data base is the gold standard of comparison, and is made from a very limited number of “presumably healthy subjects”. Each patient is compared with that gold standard to determine if the RNFL measure is normal or abnormal. Current method is not accurate and generalizable as there's an overlap between normal RNFL and abnormal RNFL thickness contour. In our proposed method, each person serves as his own reference and the comparison will be made between the current measured RNFL thickness and his “predicted” personalized undamaged RNFL thickness contour that is made possible by using a GAN model.

### BRIEF DESCRIPTION OF DRAWINGS

FIG. 1. Correspondence of near infra-red (INR) En Face image of optic nerve and peripapillary nerve fiber layer as well as peripapillary blood vessels, OCT B-scan of the same region, and the OCT-generated RNFL thickness contour/profile.

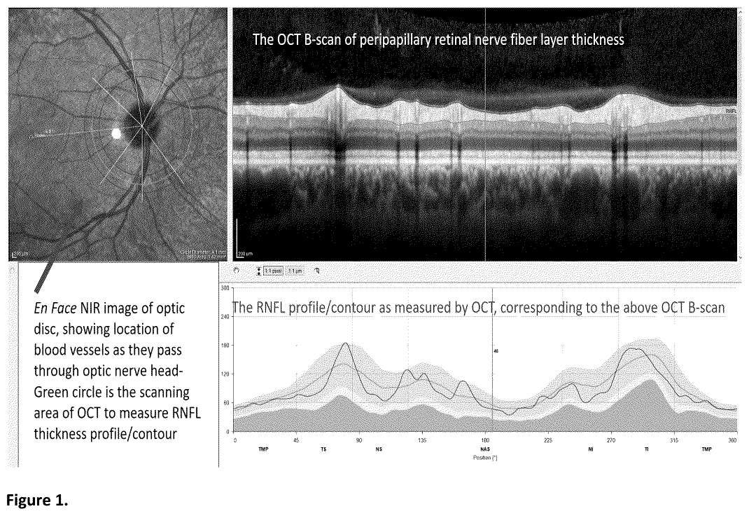

FIG. 2. Correspondence of the registered peripapillary blood vessels in an INR En Face image with their counterparts in OCT B-scan.

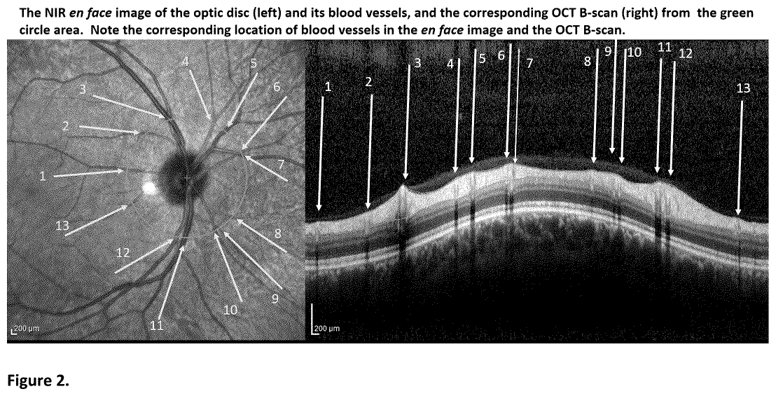

FIG. 3. OCT B-scan of the peripapillary RNFL thickness (up) with its corresponding RNFL thickness profile/contour (down). As is seen, the location of blood vessels directly influences the peaks in RNFL thickness profile/contour.

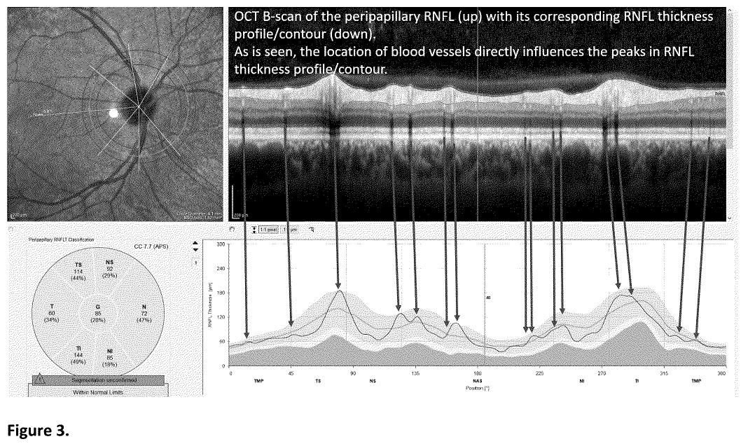

FIG. 4. X=source: shows the location, number, and the size of blood vessels. Images are produced from random selection of parameters. Y=expected: contour made by part 1 of the computer generated images, using an internal function of python MatPlot library. X and Y are made by python codes in a separate independent program.

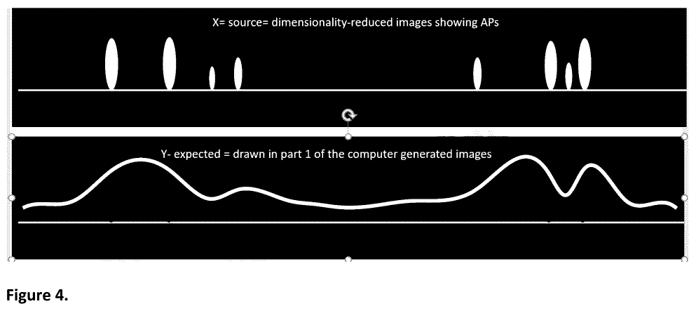

FIG. 5. Source (X), expected (Y), and generated images. Generated: RNFL thickness contour/profile made/predicted by the part 2 computer generated images, using GAN. GAN takes X as input, and generates the “generated” as its output. The comparison between the Y (=expected, made independently in part 1) and the generated (=GAN generated images in part 2, based on X input) yields a MAE of 0.021.

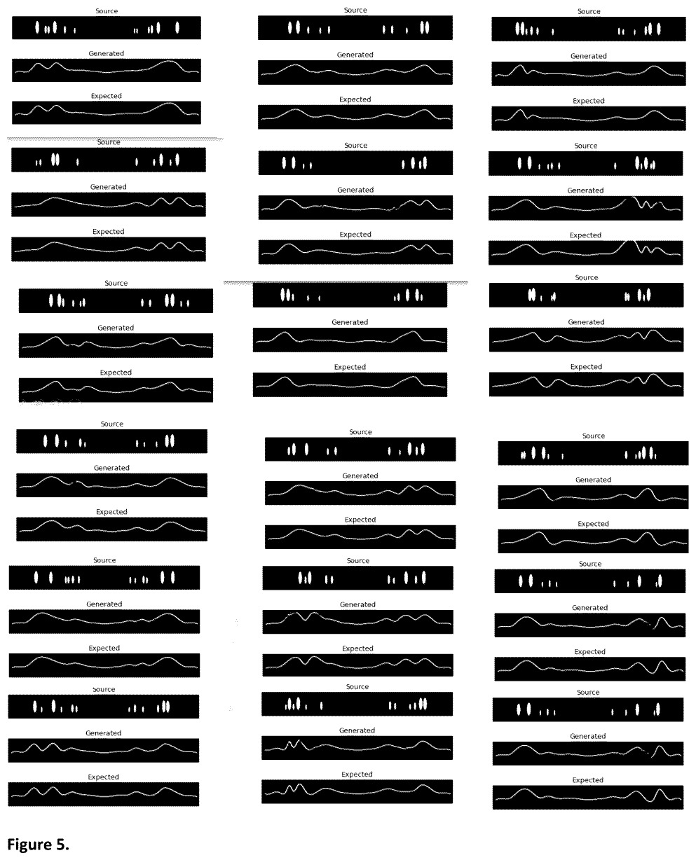

### DETAILED DESCRIPTION OF THE INVENTION

This is a proof of concept project. This invention is about using GAN to predict RNFL thickness contour/profile in OCT imaging. Therefore, this invention has two parts: how to use a GAN, and how to use OCT B-scan imaging. All images used in this project are computer generated. No human data is used in this invention.

Currently, the normative data base is the gold standard of comparison for labeling a quantifiable measure in medicine. RNFL thickness contour/profile is widely used as an objective way of diagnosing/surveillance of glaucoma. A patient's measure of RNFL thickness is compared with that of the “normative data base” to yield the label of normality vs. abnormality.

There are multiple problems with this use of normative data base in detection of abnormality using RNFL thickness contour:
a. Different brands of OCT machines have different database (from different population), therefore, a patient may be considered “within normal limits” with one OCT machine and stays “outside normal limits” when imaged by another OCT machine.
b. The data base of OCT machines is very limited (˜500 cases in the Zeiss OCT, which has by far the largest database), and cannot represent the general population.
c. Continuous improvements in hardware as well as the software makes comparison of the results over the years to be inaccurate.
d. Not every patient has historical access to the same OCT machine, and often times patients have OCT RNFL thickness measure from different machines, making comparison impossible.

Therefore, there is a real need to have “personalized” RNFL thickness contour/profile in which any patient can be accurately compared with his own normal healthy undamaged value of RNFL thickness which potentially overcomes all the shortcomings of currently using normative data base.

Based on a published article (Hood D C et al. Blood vessel contributions to retinal nerve fiber layer thickness profiles measured with optical coherence tomography. J Glaucoma. 2008 October-November; 17(7):519-28) and expanding on its concept, we hypothesized that the contour of RNFL thickness can be predicted by knowing the anatomical parameters of peripapillary number of blood vessels, blood vessel sizes, and blood vessel location.

This project had two major parts: a. computer generated images, and b. making a GAN model which can “predict” the RNFL thickness contour based on the input of the original images.

The computer generated images (part a.) have two parts: 1. Python coded dimensionality-reduced image production (X) containing only the relevant data from an OCT B-scan: the relevant data are the number, size, and location of peripapillary blood vessels. That is, we call such images as “dimensionality-reduced” images, as the rest of the OCT B-scan image are not relevant/used for this project. 2. Using an internal function of the MatPlotLib of python, a contour was made on the X images, to yield the “predicted/expected” Y images. This part of image creation to produce the needed images for the GAN model was done completely independent from the GAN model creation.

GAN is a deep learning method of artificial intelligence. GAN has two main parts: the generator and the discriminator. The generator has only access to the “source” input, while the discriminator can see both the “source” input and the “predicted” output, both are fed into the algorithm. The generative part produces an output based on the “source” input, and the discriminator compares the generator's output with the “predicted” output which is accessible only to discriminator. Based on this comparison, the discriminator marks the performance of the generator, and this interaction goes on till the discriminator cannot find any difference between the output of the generator and its “predicted” output. In our model, we provided the GAN with a “source” made from APs (peripapillary blood vessel size, location and number) and an “expected” contour made from an internal function of MatPlotLib of python. The GAN used one-thousand “source” and “expected” images for training and one-hundred of “unseen” images for prediction. It achieved MAE of 0.021.

Such bridging between the potential applications of a GAN (to “create” image/contour), currently available technology of OCT (to “measure and quantify” APs), and novel use of the knowledge of the anatomy of the eye is the core of this invention.

1. GAN method of deep learning artificial intelligence is capable of accurately predicting the personalized RNFL thickness contour/profile based on anatomical parameters of peripapillary blood vessel size, number, and location, using OCT images.
